(19) United States
(12) Patent Application Publication
(10) Pub. No.: US 2004/0067543 A1
(43) Pub. Date: Apr. 8, 2004

(54) Multi-disciplinary approach to validating or identifying targets using an in vivo system

(76) Inventors: Patrick Y. Lu, Gaithersburg, MD (US); Puthupparampil Scaria, Montgomery Village, MD (US); Martin C. Woodle, Bethesda, MD (US)

Correspondence Address: HELLER EHRMAN WHITE & MCAULIFFE LLP, 1666 K STREET,NW, SUITE 300, WASHINGTON, DC, 20006, US

(21) Appl. No.: 10/610,821

(22) Filed: Jul. 2, 2003

Related U.S. Application Data
(63) Continuation of application No. 10/263,470, filed on Oct. 3, 2002, now abandoned.

(51) Int. Cl. C12Q 1/68; G01N 33/574

(52) U.S. Cl. 435/7.23; 435/6

(57) ABSTRACT

The present invention embodies a multi-disciplinary approach to validate or identify targets involved in any given biological process or pathway, such as an immune response, or progression or regression of disease. By introducing a target(s) with, e.g., gene delivery vector(s) or other foreign substance(s) to an in vivo system and by integrating, for example, pathological, pharmacological, bioassay, microarray and bioinformatics data obtained from the in vivo system, the present inventors are able to (1) identify one or more targets, e.g., genes, that are involved in a biological pathway of interest, (2) implement these identified targets for further analysis of the biological process or pathway and (3) provide a scalable approach in vivo for potential large quantities of target(s) discovery and validation. This process can be used in any number of applications, including the identification of agonists and antagonists to a biological process or pathway, which can lead to drugs and vaccine discovery.

### BACKGROUND

The current practice of high throughput screening in a variety of in vitro assays is limited in that it yields only preliminary results of gene function and drug target hits. These results are indirect and often fail to find targets that are involved in interactions with tissues and cells unique to a particular disease. For example, the use of gene function analysis of disease tissues, or more recently tissues from different states of disease progression, relative to normal or healthy tissues reveals functions associated with formation of disease and with progression of disease, i.e. identification of disease causing genes. Such information is most relevant for diagnosis and preventative medicine but is less informative for functions useful for treatment as these functions are frequently different from those leading to the disease.

The nature of in vivo studies has limited such use to confirming the “true value” of hits. Unfortunately, even for this limited purpose, availability of reliable data still has been limiting. Nonetheless, data derived from in vivo studies yield the most accurate or true validation of drug targets. Once targets are truly validated, then conventional drug discovery techniques become an available option for more efficient drug production. Accordingly, there remains a pressing need to efficiently utilize in vivo systems for purposes of target discovery validation, to circumvent the shortcoming of in vitro applications.

### SUMMARY OF THE INVENTION

The present invention takes advantage of delivering genes or drugs or combinations of genes and drugs directly to in vivo system, or manipulating the in vivo system in other manners so as to achieve at least two different in vivo states differing in the manner that is that desired by a treatment, without the need for in vitro screens. The pathway analysis of these differing in vivo states is used to identify genes and proteins associated with the biological condition or process provided for by the system. Finally, the identified genes and proteins are rapidly validated for control of the biological condition or process provided by delivery of the gene or polynucleotide inhibitor into the in vivo system so as to express the gene or protein and observe its controlling effect. This scalable method can be used to obtain validated drug target hits. A hit can support multiple modalities of therapeutic intervention such as traditional small molecule drug discovery, conventional biotechnology protein drug development, antibody drug development, and gene expression therapeutics.

The invention provides methods of discovering a candidate target involved in the evolution of a biological pathway, including the steps of: introducing a target or targets of known or speculative function into an in vivo system; allowing the in vivo system to function in order to allow for at least partial evolution of the biological pathway; and subjecting a biological sample obtained from the in vivo system to two or more analyses selected from the group consisting of pathological, pharmacological, bioassay, microarray and bioinformatics analyses, wherein the analyses reveal one or more candidate targets involved in the evolution of the biological pathway. This method also may include introducing the one or more revealed candidate targets into an in vivo system; allowing the in vivo system to function; and subjecting a biological sample from the system to the foregoing analytical processes, in order to identify further targets involved in the evolution the biological pathway.

The invention also provides a method of validating a candidate target involved in the evolution of a biological pathway, including the steps of: introducing a polynucleotide derived from the sequence of the candidate target into a first in vivo system; allowing the first in vivo system to function in order to allow for at least partial evolution of the biological pathway; subjecting the biological sample to two or more analyses selected from the group consisting of pathological, pharmacological, bioassay, microarray and bioinformatics analyses, wherein the analyses reveal useful data; and comparing the useful data to data obtained from a control in vivo model, wherein the comparison step reveals whether the polynucleotide and thus candidate target is involved in the evolution of the biological pathway.

In addition, the invention provides targets that are identified by these and other methods described herein.

More specifically, the invention provides a method of discovering a candidate target involved in the evolution of a biological pathway, comprising (a) introducing a target or targets of known or speculative function into an in vivo system; (b) allowing the in vivo system to function in order to allow for at least partial evolution of said biological pathway; and (c) subjecting a biological sample obtained from the in vivo system to two or more analyses selected from the group consisting of pathological, pharmacological, bioassay, microarray and bioinformatics analyses, where the analyses reveal one or more candidate targets involved in the evolution of the biological pathway. The method may further comprise (d) introducing the one or more revealed candidate targets into an in vivo system; and (e) repeating steps (b) and (c) in order to identify further targets involved in the evolution of the biological pathway. Step (e) may optionally further comprise introducing the target or targets of known or speculative function into the in vivo system. Step (e) also may reveal a further candidate target or targets involved in the further evolution of the biological pathway. Step (e) also may comprise subjecting the sample to three, four, or five or more analyses selected from the group consisting of pathological, pharmacological, bioassay, microarray and bioinformatics analyses, where the analyses reveal one or more candidate targets involved in the evolution of the biological pathway.

The method may optionally further comprise (f) introducing a further candidate target or targets into an in vivo system; and (g) repeating steps (b) and (c) in order to identify still further targets involved in the still further evolution of the biological pathway.

In one embodiment the known target and candidate target may be genes. In another embodiment, the known target or targets may be introduced to the in vivo system through a vector system. The in vivo system may be a mammalian system.

In another embodiment, step (a) may further involve introducing into the in vivo system a reagent selected from the group consisting of drugs and bacterial toxins.

In a further embodiment, there is provided a method of validating a candidate target involved in the evolution of a biological pathway, comprising: (a) introducing a DNA sequence into a first in vivo system; (b) allowing the first in vivo system to function in order to allow for at least partial evolution of the biological pathway; (c) subjecting the Biological sample to two or more analyses selected from the group consisting of pathological, pharmacological, bioassay, microarray and bioinformatics analyses, wherein the analyses reveal useful data; and (d) comparing the useful data to data obtained from a control in vivo model, where the comparison step reveals whether the DNA sequence is involved in the evolution of the biological pathway.

The invention further provides targets identified by the methods described above.

### BRIEF DESCRIPTION OF THE DRAWINGS

FIG. 1 is a schematic representation of using the methods of the invention to validate a target.

FIG. 2 is a schematic representation of using the methods of the invention to discover one or more targets.

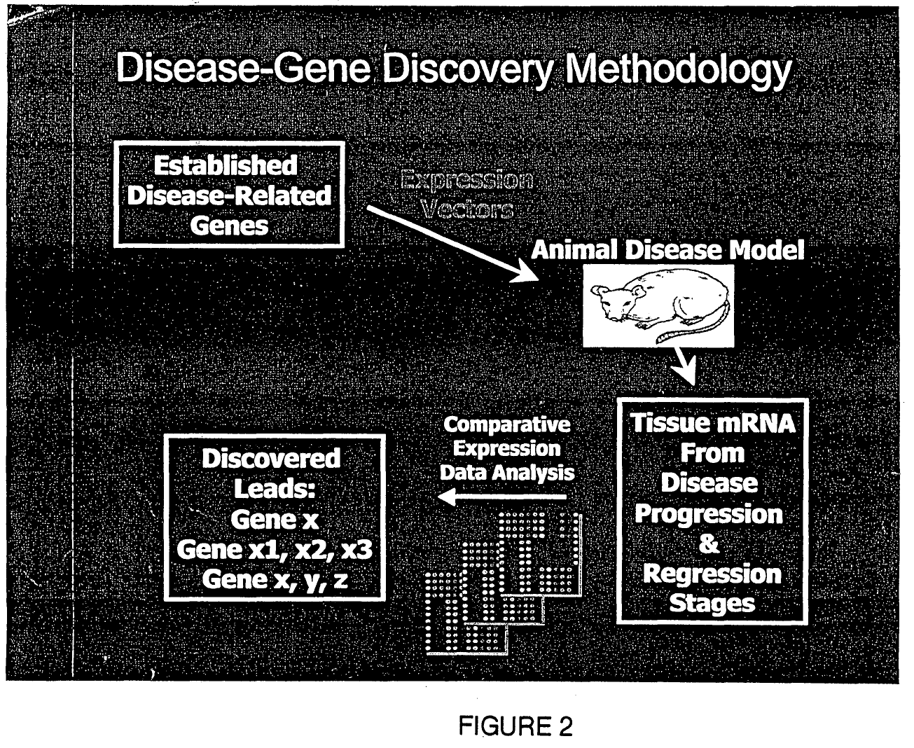

FIG. 3 shows growth curves of tumors treated with expressing plasmid DNA. The assay discriminates candidate drug targets according to their ability to single handedly enhance tumor growth or inhibit tumor growth or have no significant effect on tumor growth. All expression constructs contained a CMV promoter and a corresponding transgene. The constructs were verified by sequencing and cell culture. Each gene target was directly injected into 6 tumors on 3 mice.

FIG. 4 shows growth curves of tumors treated with expressing plasmid DNA.

FIG. 5 summarizes the microarray analysis carried out in Example 4.

FIG. 6 lists known targets identified using the methods of the invention.

FIG. 7 summarizes the novel targets identified using the methods of the invention.

FIG. 8 summarizes the types of targets identified using the methods of the invention.

### DETAILED DESCRIPTION OF THE INVENTION

The present invention provides novel multi-disciplinary methods to validate or identify targets involved in any given biological process or pathway. The methods entail manipulating the in vivo system in a manner so as to achieve at least two different in vivo states differing in the process or condition that is that desired by a treatment, then analyzing a combination of pathological, pharmacological, bioassay, microarray and bioinformatics data obtained from the in vivo system. Manipulation of the in vivo system can be achieved by several methods including 1) introducing a gene for a known target to the in vivo system so as to achieve expression of the target protein, 2) introducing a known protein target to the in vivo system, 3) introducing a drug to the in vivo system, 4) altering the environmental conditions of the in vivo system, and 5) combinations of these so as to achieve the pharmacological effect on the in vivo system. The analysis then provides genes and proteins that associate with the pharmacological effect. The analysis also informs the selection of a further molecule or target which is introduced into the in vivo system, followed by another round of analysis of pathological, pharmacological, bioassay, microarray and bioinformatics data. This iterative approach provides a rigorous assessment of the validity or usefulness of a particular target. This invention can be used in a wide variety of applications, as described herein.

Where it is desired to validate a suspected target, for example as a target for drug intervention, the invention contemplates introducing a target of an unknown or speculative function into an in vivo system, followed by the multi-disciplinary analysis described above, which reveals a gene expression pattern in the system. By combining this expression analysis with a “control” system, the unknown or speculative function of a target is then associated with a particular function, disease, or physiological pathway; that is, the target can be “validated.”

When it is desired to discover new or candidate targets involved in a particular biological process or pathway, a target preferably of a known function is introduced into an in vivo system and—after allowing the biological process or pathway to at least partially evolve in the in vivo system—a multi-disciplinary analysis is carried out. This analysis reveals gene expression and protein pattern indicating those targets, e.g., genes and proteins, that are involved in the further evolution of that biological process or pathway. These newly identified targets then can be introduced into an in vivo system to further study their effect on the evolution of the process or pathway, as well as to identify additional targets involved in the continued evolution thereof.

One example of this approach to studying disease pathways is to use gene delivery methods to either (i) express a known member of a disease-linked pathway, or (ii) to suppress expression of a known member of the pathway, thereby perturbing the disease pathway. An analysis of the type described herein is applied to other members of the pathway, thereby providing important insights into the pathway.

### Selecting a Set of Disease States

As an initial step, an in vivo system is manipulated to achieve at least two different states differing in the manner that is that desired by a treatment. For example cancer is often characterized by excessive growth rates such that achieving tumor tissues with unperturbed growth rate and other tumor tissues with stimulated and inhibited growth rates provides tissues of different states differing in the manner desired by a treatment. Another example is in arthritis where the disease is characterized in some instances by inflamed joints such that achieving joints with unimpeded inflammation is one state and another state is achieving joints with diminishing inflammation and yet another state is achieving joints with exacerbated inflammation.

In one embodiment, one or more targets is selected to be introduced into an in vivo system. The function or characterization of the target may be known, speculative or unknown, for example, depending on the manner in which the target will be used. As used herein, a target can mean: a polypeptide or protein encoded by a DNA molecule, the DNA molecule itself, or an RNA molecule derived therefrom. In another embodiment, one or more drugs are selected to be introduced into an in vivo system.

### Target Validation

In one aspect, the invention provides a method of validating a target whose function or method of action is speculative or partially or completely unknown. Any speculative, unknown or otherwise potential target can be used in the present invention. For instance, the target can be a full-length gene, such as a genomic DNA sequence, or a cDNA sequence. The target preferably has a suspected involvement in a particular biological pathway or process, or with a class of disease or disorders. However, a speculative function is not a necessary prerequisite for use in the instant invention.

One method of validating a target, according to the invention, involves introducing that target to an in vivo system (optionally in combination with other reagents), and deriving bio-analytical data from the resulting perturbed system. Those bio-analytical data are then compared, as further described herein, to data obtained from a known control. The control preferably is the best available positive control. For example, if a target is suspected of involvement in a particular disease, then the control preferably is a related in vivo disease model that has introduced into it a gene(s) that is known to be involved in a pathway of the investigated disease. Alternatively, no target is induced to the in vivo system, but instead the studied in vivo system contains a mutation, such as a knock-out mouse.

For example, if a potential target is suspected of being involved in the progression or regression of rheumatoid arthritis, then the potential target is introduced into an in vivo system having arthritic characteristics and the in vivo system is subjected to analysis, e.g., a multi-disciplinary gene expression analysis, after a period of time. Methods of gene expression analysis are well known in the art and include, for example, nucleic acid microarray analysis. Suitable microarrays are commercially available.

The derived data then can be compared against data obtained from a control in vivo system having arthritic characteristics, which has introduced into it one or more genes having an established role in an arthritic pathway. Models for rheumatoid arthritis are known in the art. See, for example, Henderson, Mechanisms and Models in Rheumatoid Arthritis, (Academic Press, 1995). The comparison of data between the potential target and the control reveals information such as the ability of the target to up-regulate or down-regulate genes that are involved in the progression or regression of the investigated disease.

In another example, a suspected tumor target can be introduced into a tumor model, such as a tumor-bearing nude mouse, and the effects on the tumor can be observed, for example, by measuring change in tumor size. Other methods of measuring the effects on the model are known in the art, for example, by measuring metastatic potential by assaying matrix metalloprotease activity.

### Target Discovery

In another aspect, the invention provides a method of discovering one or more novel targets that are involved in any given biological process or pathway, such as an immune response, or progression or regression of disease. In general, this methodology entails introducing into an in vivo system one or more known, i.e., characterized, genes (optionally in combination with other reagents). Thereafter, the system is allowed to progress through at least a partial evolution of a biological process or pathway of interest, after which at least a portion of the in vivo system can be subject to a multi-disciplinary pathway analysis. The analysis may utilize measurements of gene expression. The biological pathway or process may related to, for example, tumor progression or regression, a rheumatoid arthritis pathway, any degenerative disease, or an immune response.

The pathway analysis of the in vivo system may reveal that one or more genes are expressed at a biologically significant level, such as a heightened expression level, or it may reveal that one or more proteins are phosphorylated at a biologically significant level. The analysis further may reveal a particular stage in the biological process or pathway when expression occurs at a biologically significant level. By identifying potentially significant genes and/or proteins from this analysis, a skilled worker then can select one or more genes and introduce these genes as newly discovered targets into an in vivo system, either individually or in randomized combinations—optionally with one or more reagents, such as a drug or biological toxin.

The in vivo system, having introduced therein a newly discovered target(s), can be subjected to a similar multi-disciplinary expression analysis, after waiting a period of time that allows for further evolution of an investigated biological process or pathway. This methodology of discovering new targets, based on expression analysis of an in vivo system, can be repeated as necessary in order to characterize the evolution of a particular pathway or process and identify reagents that are capable of antagonizing or agonizing the progression of the pathway or process.

As noted, the present invention can be used to discover targets in any number of biological pathways or processes. For example, a regimen can be carried out to deduce targets involved in the development of rheumatoid arthritis. Defined genes in this pathway, such as IL-10 and IL-11, can be administered to an in vivo system either alone or in combination. The in vivo system can be, for example, a DBA/1LacJ Mouse having collagen induced rheumatoid arthritis. After a period of time (either pre-determined or randomized), a tissue sample from the in vivo system can be subject to a multi-disciplinary expression analysis. If, for instance, the expression analysis reveals biologically significant changes in expression levels of a particular gene or genes during a period of arthritic regression, then the identified gene(s) can be further analyzed by repeating the foregoing process. This process can be carried out to study the progression or regression of different types of cancers, as well as an in vivo system's immune response to an antigen, such as in the development of a vaccine.

In one aspect, the methods of the invention can employ at least two targets that are known to perturb an in vivo model system in differing or opposite fashion. For example, in a mouse tumor model, different mice can be treated with known tumor-enhancing and known tumor-inhibiting agents, for example by gene delivery of nucleic acids encoding tumor-enhancing and tumor-inhibiting agents. After evolution of the in vivo system the gene expression can be studied for the tumors treated with the tumor-enhancing and tumor-inhibiting agents. These results are be compared to untreated tumors (where the in vivo system has evolved unperturbed) and genes having altered expression can be identified. Such genes are potential targets that can be further studied by introducing the genes into the same or a different in vivo system. Of particular interest are genes that show enhanced expression under the influence of one perturbation, and decreased expression under the influence of the different or opposite perturbation. For example, in the tumor model, genes that show enhanced expression compared to control upon perturbation with a tumor enhancer and decreased expression compared to control upon perturbation with a tumor inhibitor are or particular, though not exclusive interest. Similarly, genes that show decreased expression compared to control upon perturbation with a tumor enhancer and increased expression compared to control upon perturbation with a tumor inhibitor are also of particular, though not exclusive interest.

### Choosing an in Vivo System

The present invention contemplates the use of many types of in vivo systems. Preferably, the in vivo system satisfies the following characteristics: (i) satisfactory uptake or integration of a target that is introduced into the system; (ii) satisfactory expression or presentation of the target in the system, and (iii) preferably the system exhibits a biological or physiological response to the expression or presentation of the target that is indicative of a human response to the expression or presentation of that target, i.e. the response in the in vivo system is an accurate model system for human responses.

In one embodiment, the system is a mammalian system, such as a rodent, porcine, feline, canine, ovine, or bovine. The skilled artisan will recognize that other mammalian systems may also be used. In a preferred embodiment, the in vivo system is a mouse. The mouse can be, for instance, a DBA/1LacJ mouse, a SCID mouse, nude mouse, or Balb/c Mouse. The skilled artisan with recognize that large numbers of mouse strains are commercially available and are suitable for use in the present invention. Suitable mouse strains can be obtained from, for example, The Jackson Laboratory, Bar Harbor, Me., which also provides large amounts of genotypic and phenotypic information regarding these mice.

### Introducing a Target to an in Vivo System

The target can be introduced into the in vivo system through a vector, such as a plasmid or a virus. The vector can be viral, non-viral or a hybrid, e.g. a combination of viral nucleic acid and synthetic reagents, or a polynucleotide in combination with physical delivery, e.g. pressure or electric field, and optionally involving physical methods of enhanced delivery. In accordance with the present invention, a target of interest is cloned into a vector, using conventional technology, for subsequent administration to an in vivo system. Suitable vectors are known in the art. The invention contemplates the use of any conventional vectors systems, including non-viral vector systems and viral vector systems, such as adeno virus, adeno-associated virus, retro virus, lenti virus, HSV, alfavirus, SV40, and EBV systems (see, e.g. Hitt, et al. 1997: Human Adenovirus Vectors for Gene Transfer into Mammalian cells. Gene Therapy, Advances in Pharmacology. Academic Press; Giorgio P. et al. 1997: Cytokine Gene Transduction in the Immunotherapy. Gene Therapy, Advances in Pharmacology. Academic Press; Christopher, B. et al. 1999: Retroviral Vector Design for Cancer Gene Therapy. Gene Therapy of Cancer. Academic Press; Huang, L. et al. 1999: Nonviral Vectors for Gene Therapy. Academic Press; Lowrie, D. and R. Whalen, 2000: DNA Vaccines: methods and protocols. Humana Press).

A non-viral vector can include a nucleic acid sequence coding for the production of protein factors. The nucleic acid sequence can be in circular form or linear form and may be derived from a viral genome. A non-viral vector also may include synthetic reagents, for example, lipids or liposomes (e.g., cationic lipids like DOTAP, DOTMA, DDAB or a mixture of cationic lipids with helper lipids like DOPE or Cholesterol), polymers (e.g., cationic polymers such as polylysine, branched polyethyleneimine (PEI), linear PEI, dendrimers), polypeptides (e.g., polylysine, polyarginine, polyomithine, polyhistidine, co-polypeptides of lysine and histidine, arginine and histidine, ornithine and histidine), peptides (e.g., Histone H2A and TAT protein), and polymer conjugates (e.g., conjugates of cationic polymers, hydrophilic polymers and targeting ligands).

The vector optionally can be administered to an in vivo system, via physical methodology selected from the group consisting of the application of electric field, coated bead bombardment, the application of hydrostatic pressure, and other physical methods. In the context of polymer conjugates, cationic polymers include: polylysine, branched PEI, linear PEI, copolymers of lysine and histidine; hydrophilic polymers include: polyethylene glycol, polyoxazalone, fleximer; and ligands include: peptide ligands, sugar ligands, antibodies, and single chain antibodies.

In a mammalian system, a number of viral-based expression systems may be utilized. In cases where an adenovirus is used as an expression vector, the coding sequence of interest, i.e., the target, may be inserted into an adenovirus transcription/translation control complex, such as the major late promoter and tripartite leader sequence. This chimeric gene then may be inserted into the adenovirus genome. Insertion into a non-essential region of the viral genome (e.g., region E1 or E3) will result in a recombinant virus that is viable and capable of expressing a target protein in infected hosts (e.g., See Logan et al., 1984, Proc. Natl. Acad. Sci. USA 81:3655-3659). In one embodiment, a cDNA sequence encoding the full-length open reading frames of a target can be ligated into pCMVB, replacing the β-galactosidase gene such that cDNA expression is driven by the CMV promoter (Alam, 1990, Anal. Biochem. 188: 245-254; MacGregor et al., 1989, Nucl. Acids Res. 17: 2365; Norton et al. 1985, Mol. Cell. Biol. 5: 281).

According to the gene target or targets and tissue or cell types involved, a particular type of expression vector may be selected for efficient gene delivery and being suitable to the disease model. Tissue-specific delivery (e.g., muscle, neuronal or bone) may involve a specific type of vector; for instance, naked DNA plasmid is appropriate for delivery to muscle tissue and AAV is efficient for neuron transduction in brain cells. Adenovirus vectors exhibit a preferential attraction to liver and lung tissue. The specificities of gene delivery are associated with particular sets of gene discovery and validation. Controlled target(s) expression by transcriptional regulation of a tissue specific promoter or other induction reagents can provide a unique expression environment for target discovery and validation.

Once cloned into a vector, an effective amount of the target can be introduced into an in vivo system. As used herein, an effective amount means the minimum concentration of a target needed to produce a detectable effect of that target on an in vivo system.

### Routes of Administration and Evolution of the Investigated Pathway or Process

The present invention contemplates any conventional route of administering a gene expression vector into an in vivo system. For example, the administration can be invasive or non-invasive, local (e.g., intraperitoneal, localized injection to a joint, intra-tumoral injection) or systemic. Intravenous injection of vectors, for example, is a typical method of invasive and systemic delivery. Oral-tracheal delivery is an example of local and non-invasive administration. Non-invasive deliveries involve in all types of direct administrations through open channels of the body or trans-dermal. Invasive delivery includes the injection, electroporation and open surgery, etc. The routes for gene deliveries in this invention for target discovery and validation include all types of delivery approaches currently in practice, or that may be developed.

### Multi-Disciplinary Analysis of the Derived Data

A multi-disciplinary approach is used to analyze pathways including gene expression patterns, protein patterns, protein phosphorylation patterns, and other characterizations of an in vivo system that has been subjected to one or more targets. In this regard, the invention provides for utilizing a combination of analyses, including: pharmacological and pathological data analysis; bioassaying; microarray analysis; proteomic analysis, and bioinformatics. Preferably, an analysis according to the invention factors in each of the foregoing analytical disciplines, though the invention also contemplates using a subset of these techniques. In any event, analysis of the integrated data reveals candidate genes or other factors that are involved in the evolution of a biological pathway or process. The following criteria may be used to identify candidate targets, although the skilled artisan will recognize that other criteria are available or will become available, and may be used in the context of the present invention.

### Pathological Data

A pathological analysis includes assessing the effects of a particular target on the pathology of a disease or other biological pathway. If the in vivo system is being used to monitor the progression of a tumor, for instance, then the pathological effects of an administered target on both the tumor and on the system as-a-whole would be relevant. Such effects include any changes in animal behavior, such as fatigue or lethargy; alterations in white blood cell levels; and phenotypic changes in the tumor. Methods for determining and assessing such pathological information are known in the art.

### Pharmacological Data

A pharmacological analysis is directed to the effect of a target(s) and/or other administered reagents on the gene expression levels for genes that play a role in the studied pathway or process. By way of example, gene expression levels for a particular set of genes, e.g., TNF and TNF-R can be assessed before, during and after administration of a target or other reagent to the in vivo system. The levels of gene expression provide insight into a particular target's involvement in the up- or down-regulation of particular genes. Accordingly, it is preferable to perform pharmacological analyses at various stages of a particular pathway in order to deduce whether a target is involved in turning a gene “on” or “off.”

A pharmacological analysis more specifically may be broken down into different levels: molecular, cellular, tissue and pathological. The invention may utilize analysis at each of these levels. A pharmacological analysis at the molecular level may deduce, for example, the level of cytokine expression as a biological pathway or process evolves. In an arthritic pathway, for example, a therapeutically undesirable occurrence of inflammation would stimulate inflammatory cytokine production. Inflammation likely would also result in the recruitment of neutrophils, which is an example of pharmacological effect at the cellular level. At the tissue level in an arthritic pathway, chronic inflammation would result in tissue proliferation. And at the pathological level, inflammation, i.e., progression of the arthritic pathway, would lead to loss of cell viability and, thus, destruction of certain tissue. An assessment of the pharmacological effects of a target at any of these levels can, therefore, provide further insight into validating a target or discovering new targets involved in any given biological process or pathway.

### Microarray, Gene Expression, and Proteomic Analyses

A microarray analysis can be performed on a target that is in the form of a DNA, RNA or a polypeptide molecule. By hybridizing to or otherwise interacting with a probe moiety on an array, a target can be characterized as belonging to a particular family of genes, preferably having known function. The invention contemplates utilizing any of the foregoing microarray processes, either alone or in combination, for a target that is used in accordance with the present invention. Methods of microarray analysis of nucleic acids and of peptides, polypeptides and proteins are known in the art.

If the DNA component of a target is to be analyzed, then the target may undergo a primary screening via, for example, a commercially available microarray chip. The analyzed DNA may be obtained directly from the in vivo system; alternatively, the DNA can be obtained by reverse transcribing isolated RNA, using conventional means, to produce cDNA. Suitable chips for this type of analysis include those available from Affymetrix (Santa Clara, Calif.) and Incyte (Palo Alto, Calif.). A primary screening may reveal that the target is involved in a particular “genus” of pathways, e.g., tumor growth or arthritic pathway. The data obtained by the primary screen, accordingly, can be used to focus a secondary, or specific, microarray screen. To this end, a specific microarray can be tailored to a particular pathway, such as tumor progression or arthritis. A specific screening step is, therefore, capable of revealing more specific data about a particular target, compared to a primary screening. Off-the-shelf microarrays can be obtained commercially. Alternatively, the specific microarray may be custom made using methods that are well known in the art. See, for example, Schena (Ed.) “Microarray Biochip Technology,” (Eaton Publishing Co., 2000) and Schena (Ed.) DNA Microarrays: A Practical Approach (Practical Approach Series (Oxford University Press, 1999).

An RNA Microarray analysis can be performed on RNA obtained directly from an in vivo system or indirectly, such as by obtaining DNA from the in vivo system and transcribing the DNA into RNA in vitro. RNA arrays from a commercial suppliers, such as Clontech (Palo Alto, Calif.), can be used for this type of analysis.

Protein arrays or tissue arrays can be used to identify a target in its polypeptide form. The invention contemplates the analysis of a polypeptide that is directly obtained from an in vivo system; that is, the polypeptide is translated in vivo. In addition, a polypeptide may be translated in vitro, based on DNA or RNA obtained from the in vivo system, then undergo a protein array analysis.

Proteomic analyses can be used to identify a target in its polypeptide form and specific post-translational modification forms such as phosphorylation and glycosylation. The invention contemplates the analysis of a polypeptide that is obtained from proteomic analyses. In one embodiment, phosphorylation patterns can reveal the result of kinase and phosphorylase activity that is the target. Any proteomic analytical methods can be used in the invention including 2-D gel electrophoresis, mass spectroscopy, CIPHERGEN sample preparation methods in combination with mass spectroscopy, and other methods known to one skilled in the art of proteomic analyses.

### Bioassays

Whereas a microarray analysis can provide qualitative data, i.e., whether a particular gene is turned on or off at a particular segment of a biological process or pathway, bioassays provide quantitative data, such as the levels of gene expression at a particular stage of a process or pathway. Specific bioassays suitable for use in this context include, inter alia, Northern, Southern and Western Blotting, for analyzing a target in its DNA, RNA and polypeptide form, respectively. Such methods are well known in the art (Ausubel, et al., (eds.) 1989, CURRENT PROTOCOLS IN MOLECULAR BIOLOGY, John Wiley and Sons, Inc.).

### Bioinformatics

A bioinformatics analysis permits an expressed gene or set of genes to be identified as belonging to a particular family of genes. Preferably, this type of analysis will allow a skilled worker to group, or “cluster,” genes based on function or expression intensity, for instance. This approach may, therefore, reveal multiple genes that, in tandem, can affect the evolution of a particular biological process or pathway.

### Validating a Target Based on a Multi-Disciplinary Analysis

A combination of the foregoing analyses can reveal, with relative precision, the role a target plays in a particular biological process or pathway of an in vivo system. In this regard, the data obtained from a studied in vivo system can be compared to data obtained from a control system. A comparison of expression data may reveal, for example, that the target is involved in suppressing the expression of a gene that otherwise is expressed in the control system.

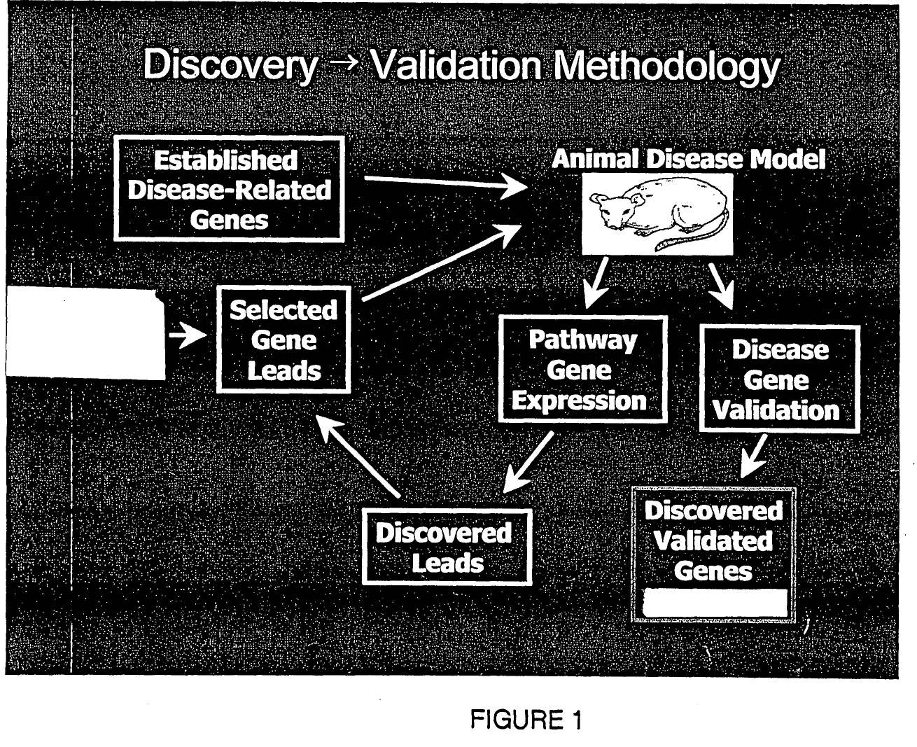

With reference to FIG. 1, a target of unknown or speculative function, i.e., a “gene lead,” can be administered to an animal disease model, such as a mouse. After a period of time, the animal model can be analyzed, and derived data can be compared with data obtained from an expression analysis of a similar animal model having had introduced thereto one or more “established” genes, i.e. a control. The data comparison can lead to the validation of a disease gene.

### Introduction of the Newly Discovered or Identified Target(s) Into an In Vivo System

In addition to revealing the function of a target, the unique combination of data obtained from an in vivo system can provide insight as to other targets that may be involved in the progression or regression of a particular pathway or process. Accordingly, the integration of data can lead to the discovery of new targets, which subsequently can undergo in vivo administration, as prescribed herein for, e.g., target validation.

Any target of known function can be used in the present invention as a tool for discovering new targets. Accordingly, the target may either stimulate or suppress the progression of a disease. If an employed target causes disease progression, one object is to identify other genes that are involved in this progression, to develop antagonists to such genes.

The target also may be an antigen that is capable of eliciting an immune response. One object in using this type of target would be to develop a vaccine having a heightened efficacy. To this end, an target antigen may be introduced into an in vivo system, followed by a multi-disciplinary expression analysis to identify one or more active genes, or targets, such as a cytokine. The target antigen then may be re-introduced to an in vivo system concomitantly with the candidate target(s), followed by another multi-disciplinary expression analysis. The analysis may produce a further heightened immune response, while revealing other candidate targets that could be re-introduced to an in vivo system in conjunction with the target antigen and other identified targets.

### EXAMPLES

The following examples are intended to be illustrative only and, thus, are not limiting.

### Example 1

### Adeno Associated Virus Vector Mediated Soluble TNF Receptor Gene Delivery Into DBA/1LacJ Mouse Collagen Induced Rheumatoid Arthritis

The disease regression should be observed at different time points and at different dosage levels, and the pathological and pharmacological readouts are collected. When RNA samples are collected from each cohort group and then subjected to the Microarray analysis, the up regulated and down regulated gene target is identified. By combining the bioassay data, Microarray data and Bioinformatics data, the potential targets for genomic drugs are identified. Those new targets are subjected to the same type of animal model again and become confirmed and validated targets following suitable analysis as described above.

### Example 2

### Adenovirus Vector Mediated GM-CSF Gene Delivery into SCID Mice

Adenovirus vector mediated GM-CSF gene delivery into SCID mice tumor model is a suitable example for immune boosting of host. The expression profiles after treatments at different dosages and different time points likely are distinct, as tumor regression occurs. The same types of sample collection as in Example I, e.g., pathological and pharmacological data, microarray and bioinformatics data provide the adequate information for the new target discovery.

### Example 3

### Validation and Elimination of Candidate Cancer-Related Targets

Summary: A gene expression plasmid DNA for each candidate target, along with controls, was delivered into xenografted MDA-MB-435 tumors in 6-week old female nude mice (Taconic, Germantown, N.Y.) in a series of injections every five days for 15 to 20 days. The tumor growth rates during this period were measured using a double-blinded protocol.

### Brief Description of Study

A group of genes was cloned into the pCI expression vector (Promega, Madison, Wis.) and delivered intratumorally into human MDA-MB-435 tumor cells xenografted in nude mice by using a histidine-lysine copolymer (see for example EP 1242051 A1, which is hereby incorporated by reference in its entirety). The product of transgene expression perturbs tumor growth in a manner dependent on the function of the individual gene. Comparison of tumor growth curves among different treatment group permitted interpretation of the biological function of the gene with regard to its ability to regulate tumor growth.

### Experimental Design

1). Establishment of MDA-MB-435/nude tumor models: 4×10⁵ MDA-MB-435 cells suspended in 30 ul of RPM1640 medium without serum were s.c. injected into nude mice. The tumors were allowed to grow until their sizes reached 50 to 150 mm³.

2). Plasmid DNA of selected candidate human genes (listed in Table 1) in the pCI expression vector was directly delivered into tumor using a histidine-lysine (HHHK) copolymer. Each tumor received 5 μg DNA mixed with 5 μg of the copolymer 4B polymer. Each group contained at least 3 mice (6 tumors). DNA injections were performed every 5 days for 20 days (4 injections).

3). The tumor size was measured in two dimensions using external calipers and the tumor volume was calculated as Volume=width²×length×0.52. Volume was measured before every DNA injection and every 5 days after last DNA injection for a period of 25 days. The survival of treated mice was closely monitored. The mouse was sacrificed when the tumor size reached 3000 mm³ or at the end of experiment.

4). The study was double-blinded to demonstrate the ability of the assay to correctly discriminate the targets according to their ability (relative to control gene and vector samples) to 1) enhance tumor growth, 2) inhibit tumor growth, or 3) have no effect on tumor growth.

### Materials and Reagents

1). Cell: MDA-MB-435 is a human breast cancer cell line and was a gift from Dr. James Mixson (University of Maryland, Baltimore). The cell line was cultured under standard conditions.

2). Plasmid DNA: The maxi-preparation of plasmid DNA was carried out using a Qiagen EndoFree Plasmid Maxi kit (Qiagen Cat# 12362) follow the protocol recommended by the manufacturers.

3). Histidine-lysine HHHK 4B polymer: The histidine-lysine copolymer was provided by Dr. James Mixson )University of Maryland) and used at 30 μg/ul.

### Results

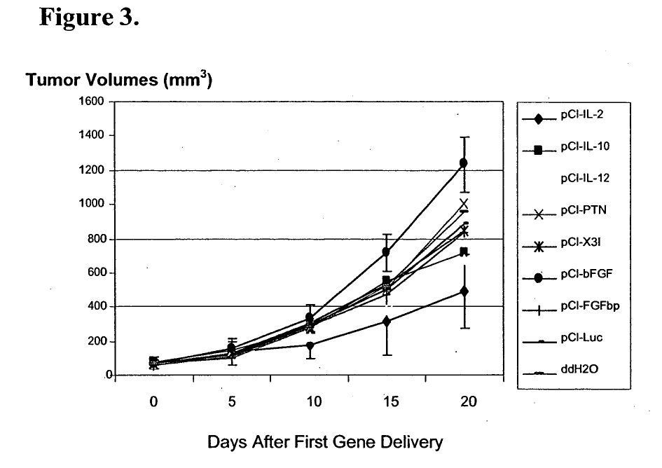

A selection of candidate genes in expression plasmids was studied alongside several well-known tumor targets, as listed in Table 1 and Table 2. The study was performed in a blinded test to demonstrate the assay could correctly discriminate between known targets, as shown in FIG. 3, according to their ability to 1) enhance tumor growth, 2) inhibit tumor growth, or 3) have no effect on tumor growth, relative to control gene and vector samples.

The known targets included in the study were correctly identified by the tumor growth rate readouts. Enhanced tumor growth was observed in the group of animals treated with plasmid DNA expressing human bFGF. Human bFGF is a well-known target that enhances tumor angiogenesis and growth and is the target for an approved cancer drug. Inhibited tumor growth was observed in the group of animal treated with plasmid DNA expressing human IL-2. Human IL-2 also is a well-known target with anti-tumor (anti-angiogenesis) activity and, in fact, is an approved drug for treating renal cell carcinoma (the immune response activity of IL-2 was not a major factor in the observed response in this assay). A luciferase reporter gene was correctly identified as having no effect on tumor growth. Comparison of the results from the study to the properties of the known targets is shown below in Table 1. The results of this study matched perfectly with the known properties of these three genes, clearly demonstrating the ability of the described methods to discriminate candidate tumor targets.

TABLE 1
Validation of Tumor Perturbation Assay

| | | | |
|---|---|---|---|
| Gene: | IL_2 | Luciferase | bFGF |
| Established property: | Inhibition | None | Enhanced |
| Exp. result: | Inhibition | None | Enhanced |

This successful demonstration was reinforced by the results obtained from five candidate targets included in the study. These targets have been previously described in the scientific literature, and some of them have been studied for many years in cell culture and various animal studies. Two of these five genes, FGF binding protein (FGFbp) and pleiotrophin (PTN), have been suggested to enhance tumor growth. The study of PTN was a good test of the power of this technology platform to eliminate weak targets. The PTN was identified by association with human breast cancer tissues, found to have protein characteristics very similar to accepted targets, and to have mechanistic activity in angiogenesis in model organisms. However, despite these many studies and apparent success as a candidate target, as can be provided by all the standard genomics, proteomics, and bioinformatics methods, it has yet to be proven as a tumor controlling target. By using the tumor-bearing animal model combined with gene delivery technique described above, it was possible to discriminate PTN from proven targets in a rapid fashion.

Similarly, two of the five candidate targets, human IL-12 and human IL-2, have been suggested to inhibit tumor angiogenesis and thereby inhibit tumor growth. While IL-10 is generally considered weaker than IL-2 for inhibition of tumor angiogenesis, IL-12 is known to be similar or stronger than IL-2. The human IL-12 gene tested in this study contains only one of its two domains providing a test of whether both domains are needed for inhibiting tumor angiogenesis. A third potential angiogenesis inhibitor of unknown structure (X) also was a candidate target. The date obtained using the methods described above data strongly suggested that this target might not be a good candidate for anti-cancer drug development.

In all cases, despite considerable scientific investigation, the five targets under study lack clear and convincing evidence of their ability to single handedly control tumor growth. The results obtained are summarized in Table 2. Further testing of these candidates in other tumor models can be performed with same technology platform to validate them or further eliminate them from consideration as drug development candidate.

TABLE 2
Tumor Perturbation Method Evaluation of Candidate Tumor Targets

| | Genes | | | | |
|---|---|---|---|---|---|
| | FGFbp | PTN | IL-12* | IL-10 | X3 |
| Hypothe- | Enhanced | Enhanced | Inhibition | Inhibition | ? |
| sized | | | | | |
| Our Result | No | No | No | No | No |
| | observed | observed | observed | observed | observed |
| | effect | effect | effect | effect | effect |

The methods described above take less than 45 days and, starting with a plasmid expression construct, makes this method an extremely effective means to discriminate candidate targets. The method rapidly determines whether genes identified from in vitro studies as having strong correlation with tumor cell proliferation have the ability to control growth of a tumor mass in a complete biological system. It will be apparent that the methods described herein permit running the assay with multiple tumor models. For example, the methods can be employed with a battery of up to four tumor models run simultaneously.

### Example 4

### Disease-Linked Target Discovery

### Tumor Perturbation

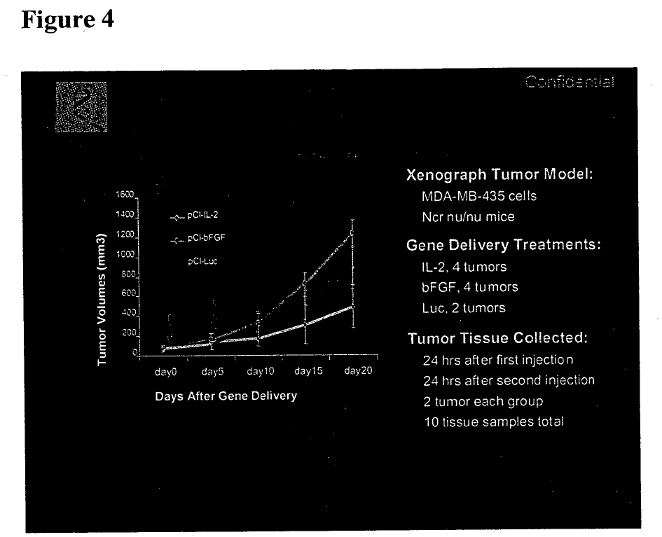

IL-2 and bFGF were selected based on the data from the target validation experiment which showed that IL-2 clearly inhibited tumor growth, and bFGF enhanced tumor growth. Mice were injected in their tumors as described in Example 3, using either 10 ug of pCI-IL-2 or pCL-bFGF plasmid, with HHHK copolymer. Injections were made every 5 days for 20 days. The results on tumor volume are shown in FIG. 4.

Based on these results it was apparent that tumor growth was not significantly impacted for about the first 5 days after the initial injection, although it was known that tumor enhancement or inhibition processes must be occurring in the tumors (based on their subsequent growth rates). Accordingly, the first and second injection points were selected for use in target discovery methods.

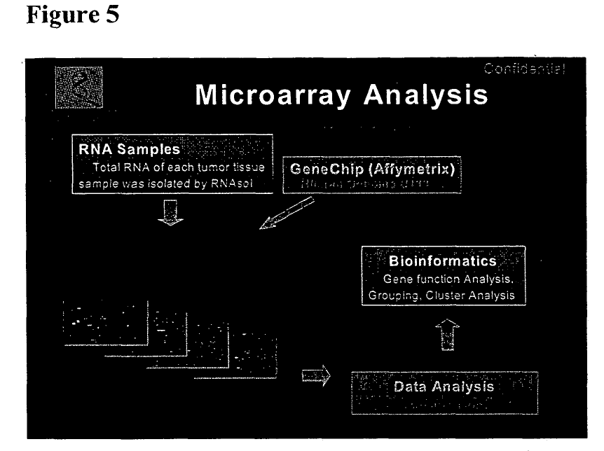

Additional mice were injected either with the IL-2, bFGF or control (luciferase) plasmid. Some mice from each group were sacrificed one day after the first injection, and the remaining mice sacrificed one day after the second injection (day 5). Tumors were removed from the mice, homogenized, and RNA prepared using standard methods. The RNA was then subjected to DNA microarray analysis (Affymetrix) using standard methods. The array data were then analyzed using standard bioinformatic methods and gene expression in the IL-2 and bFGF treated tumors was compared to the expression in the control tumors. This process is summarized in FIG. 5. This comparison was carried out at both time points. Genes whose expression was changed in either the IL-2 or bFGF-treated tumors compared to the control tumor were identified. Of particular, but not exclusive, interest were genes that exhibited enhanced expression (relative to control tumor) in the bFGF treated cells, and decreased (relative to control tumor) expression in the IL-2 treated cells. Similarly of interest were genes that exhibited enhanced expression (relative to control tumor) in the IL-2 treated cells, and decreased (relative to control tumor) expression in the bFGF treated cells. Such genes were presumably important in their effect on tumor growth or inhibition pathways.

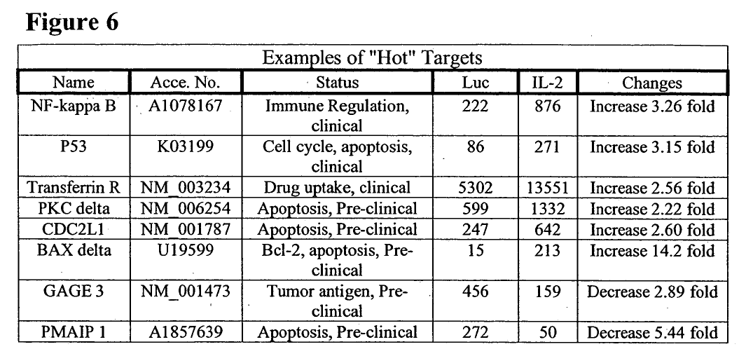

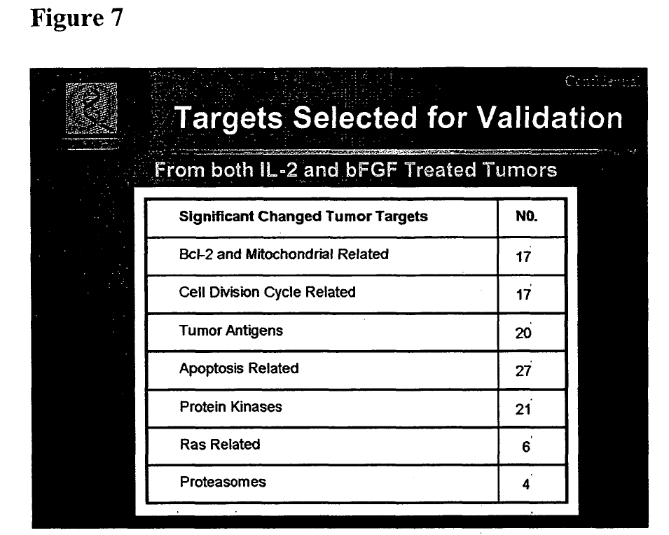

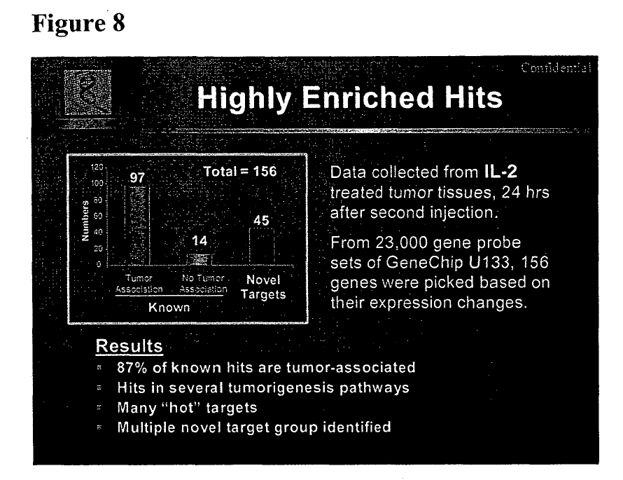

This approach identified a number of previously known and validated (“hot”) targets as shown in FIG. 6 and also identified a significant number of new targets for subsequent validation using the methods described in Example 3. These new targets are summarized in FIG. 7. FIG. 8 summarizes the results obtained using this method

### Example 5

### Tumor Growth Rate Perturbation Followed by Genomic and Proteomic Pathway Analysis for Cancer Target Discovery

Tumors implanted into animals are grown and treated so as to achieve tumors with stimulated growth rate, inhibited growth rate, and unmodified growth rate, as described in Examples 3 and 4. Human tumors are implanted into immune-compromised mice such as the nude mouse. Stimulation of tumor growth rate may be achieved by administration of agents that enhance tumor growth, for example, VEGF, bFGF, EGF, polynucleotides that result in tumor expression of these factors, or polynucleotides that result in inhibition of tumor suppressors or apoptosis inducing proteins. Inhibition of tumor growth rate is achieved by administration of agents that inhibit tumor growth including IL2 and other cytokines, tumor suppressor proteins, polynucleotides that result in tumor expression of these factors, or polynucleotides that result in inhibition of tumor growth factors or stromal tissue neovascularization. When plasmids are delivered into the tumors to stimulate or inhibit tumor growth rate, the product of transgene expression within the tumor affects the growth of tumor depending on the function of the transgene. Comparison of tumor growth curves is carried out to identify treatment groups giving stimulated, inhibited, and unaltered growth rates.

The tumors resulting from altered and unaltered growth rates, now enlarged and reduced in size relative to the unaltered growth rate tumors, are removed and processed as described in Example 4 and their gene expression proteomic profiles are determined. Additionally, a repeat of the implantation and treatment is performed, providing tumors that are destined to become enlarged or reduced in size relative to tumors with unaltered growth rate but which are as yet substantially the same size, and these tumors are removed and processed to determine their gene expression proteomic profiles. Tumors are processed, for example by snap freezing, to generate samples for measurements. Processing is performed to obtain a) thin sections for laser capture microdissection of selected tissue structures and b) samples of bulk tissue. RNA samples are collected from each cohort group and then subjected to microarray analysis, for example using oligonucleotide arrays for EST determination or cDNA arrays for gene determination. Protein samples are collected from each cohort group and then subjected to proteomic analysis, for example 2-dimensional gel electrophoresis combined with mass spectroscopy analysis of spots or CIPHERGEN sample processing and mass spectroscopy analysis.

This method identifies up regulated and down regulated genes and altered proteins in the altered tumors relative to the unaltered tumors. Bioinformatic analysis is performed using standard methods to identify novel proteins associated with the altered growth rate of tumors, i.e. novel targets. The novel targets are validated by administration of polynucleotides that increase or decrease the levels of the target in the tumor tissue. Those targets that control tumor growth rate are further validated, for example, as described in Example 3.

We claim:

1. A method of discovering at least one protein or gene associated with a process or condition of an in vivo system, comprising:
a) delivering one or more genes to the in vivo system and delivering at least one pharmacological agent to the in vivo system, wherein the one or more genes and the one or more pharmacological agents generate at least two different states of the process or condition via gene expression, and;
b) obtaining information for at least two of the different generated states by conducting at least one assay selected from the group consisting of a pathological assay, a pharmacological assay, a bioassay, a microassay and a bioinformatics data assay; and
c) selecting at least one protein, gene or other molecule that is affected by the first manipulation from the information of step b).

2. A method as described in claim 1, wherein step b) comprises measuring gene expression level from each of the different generated states and step c) comprises comparing measured gene expression level between the different generated states.

3. A method as described in claim 1, wherein the process or condition is a cancerous growth.

4. A method of discovering at least one protein or gene associated with a biological process or condition, comprising:
a) manipulating an in vivo system to generate at least two different states of the process or condition;
b) obtaining information for at least two of the different generated states by conducting at least one assay selected from the group consisting of a pathological assay, a pharmacological assay, a bioassay, a microassay and a bioinformatics data assay;
c) selecting at least one protein, gene or other molecule that is affected by the first manipulation from the information of step b);
d) manipulating an in vivo system in a manner that is expected to affect at least one protein, gene, or other molecule from a common pathway associated with the protein, gene or other molecule selected in step c) to create at least two different states of the process or condition;
e) obtaining information for at least two of the different generated states by conducting at least one assay selected from the group consisting of a pathological assay, a pharmacological assay, a bioassay, a microassay and a bioinformatics data assay; and
f) selecting at least one protein, gene or other molecule that is affected by the second manipulation from the information of step e).

5. A method as described in claim 4, wherein at least one manipulation step comprises transgenic expression of a gene.

6. A method as described in claim 4, wherein step d) comprises introducing at least one gene, protein or molecule selected from step c) into the in vivo system.

7. A method as described in claim 4, wherein the at least one gene, protein or molecule selected from step c) is a sequence specific polynucleotide inhibitor.

8. A method as described in claim 4, wherein at least three assays are conducted in step e).

9. A method as described in claim 4, wherein at least four assays are conducted in step e).

10. A method as described in claim 4, further comprising introducing a protein, gene or other molecule selected from step f) into an in vivo system.

11. A method as described in claim 4, wherein the protein, gene or other molecule selected in step f) is a gene or sequence specific polynucleotide inhibitor.

12. A method as described in claim 4, further comprising:
g) introducing the selected at least one protein, gene or other molecule from step f) into an in vivo system;
h) manipulating the in vivo system in a manner that is expected to affect at least one protein, gene, or other molecule from a common pathway associated with the protein, gene or other molecule selected in step f) to create at least two different states of the process or condition;
i) obtaining information for at least two of the different generated states by conducting at least one assay selected from the group consisting of a pathological assay, a pharmacological assay, a bioassay, a microassay and a bioinformatics data assay; and
j) selecting at least one protein, gene or other molecule that is affected by the manipulation from the information of step i).

13. A method as described in claim 4, wherein the at least one gene, protein or molecule selected from step c) is a sequence specific polynucleotide inhibitor.

14. A method as described in claim 4, wherein the in vivo system is a mammal.

15. A method as described in claim 4, wherein the manipulation of step a) comprises introducing at least one drug or bacterial toxin into the in vivo system.

16. A method as described in claim 15, wherein the introduction occurs a vector system.

17. A method as described in claim 4, wherein the assay used in at least step b) or step e) is a gene expression analysis.

18. A method as described in claim 17, wherein the gene expression analysis is carried out with a nucleic acid microarray.

19. A method as described in claim 17, wherein the gene expression analysis is carried out by proteomic analysis.

20. A method as described in claim 4, wherein the in vivo system of a) and the in vivo system of d) are animals that contain tumors.
